(19) United States
(12) Patent Application Publication
(10) Pub. No.: US 2026/0345172 A1
(43) Pub. Date: Nov. 5, 2026

(54) Bypass Graft Friendly Exclusion Clip

(71) Applicant: AtriCure, Inc., Mason, OH (US)

(72) Inventors: Frank Fago, Mason, OH (US); Tim McGill, Mason, OH (US)

(73) Assignee: AtriCure, Inc., Mason, OH (US)

(21) Appl. No.: 19/629,685

(22) Filed: Mar. 26, 2026

Related U.S. Application Data
(60) Provisional application No. 63/797,539, filed on Apr. 30, 2025.
(60) Provisional application No. 63/968,008, filed on Jan. 26, 2026.

(51) Int. Cl. A61B 17/122

(52) U.S. Cl. CPC A61B 17/1227

(57) ABSTRACT

An exclusion device comprises a pair of elongated beams, each of the beams including a tissue engagement surface and an outer surface, the tissue engagement surfaces facing each other, wherein the pair of elongated beams each have a dominant dimension in a longitudinal direction; at least one spring coupled to the pair of elongated beams and biasing the beams toward each other, wherein the tissue engagement surfaces include at least one of a porous surface, a textured surface, or a textile. The porous surface comprises a plurality of holes, and the textured surface comprises a plurality of a linear pattern, a crosshatched pattern, ridges, checkering, or bumps. The textile comprises at least one of a nonwoven fabric selected from polyethylene terephthalate (PET) yarn, thermoplastic polyurethane (TPU), polycaprolactone (PCL), and poly(lactic-co-glycolic acid) (PLGA).

### CROSS-REFERENCE TO RELATED APPLICATIONS

This application claims the benefit of priority to U.S. Provisional Patent Application No. 63/968,008, filed Jan. 26, 2026, and U.S. Provisional Patent Application No. 63/797,539, filed Apr. 30, 2025, the disclosures of which are incorporated by reference herein in their entirety.

### BACKGROUND

The present disclosure relates generally to methods and devices for use with surgical applications, and more specifically to equipment that may be used during minimally invasive surgical procedures.

One of the largest market segments for a popular left atrial appendage (LAA) exclusion clip (exclusion device) is its use concomitantly with coronary artery bypass grafting (CABG) procedures. The CABG procedures place the exclusion device very close to or in contact with the new coronary artery bypass grafts. However, the current exclusion clip designs lack specific considerations tailored for these surgeries. What is needed is an LAA exclusion clip that not only preserves those features which promote integration with cardiac tissue at the contact surface of the exclusion clip but also incorporates strategically designed portions or sections to inhibit the unintended growth of the bypass graft into the exclusion clip. This dual functionality would enhance the clip's effectiveness and safety profile in CABG applications.

### SUMMARY

In a first embodiment of the invention, an exclusion device comprises a pair of elongated beams each of the elongated beams including a tissue engagement surface and an outer surface. The tissue engagement surfaces face each other, and the pair of elongated beams each have a dominant dimension in a longitudinal direction. At least one spring is coupled to the pair of elongated beams and the spring biases the beams toward each other. The tissue engagement surfaces include at least one of a porous surface, a textured surface, or a biocompatible textile.

In a first detailed arrangement, the porous tissue engagement surfaces may include a plurality of holes. The holes are sized to support the ingrowth of tissue and secure the exclusion device into the tissue.

In a second detailed arrangement, the textured surface may include one of a linear pattern, a crosshatched pattern, ridges, checkering, or bumps. Each of the textures support the ingrowth of tissue and secures the exclusion device into the tissue.

In a third detailed arrangement, the textile may include at least one of a nonwoven fabric selected from polyethylene terephthalate (PET) yarn, thermoplastic polyurethane (TPU), polycaprolactone (PCL), and poly(lactic-co-glycolic acid) (PLGA). Each of the provided textiles is biocompatible and supports the ingrowth of tissue to secure the exclusion device into the tissue.

In a fourth detailed arrangement, the tissue engagement surfaces may include a convex profile extending in the longitudinal direction and including a first longitudinal apex. The convex profiles may provide a more focused location on the tissue engagement surfaces for engaging coronary tissue.

In a fifth detailed arrangement, the at least one spring may be configured to apply a uniform closing force along the first and second tissue engaging surfaces. A single spring may be at a proximal end of the beams, or one or more springs may couple the beams at each of the proximal and distal ends of the beams.

In a seventh detailed arrangement, the texturized tissue engagement surfaces may be interlocking. The interlocking surfaces may serve to keep the beams of the exclusion device in alignment and prevent the beams from moving and disturbing the tissue.

### BRIEF DESCRIPTION OF THE DRAWINGS

FIG. 1 is a front view of a human heart presenting common coronary artery bypass graft sites.

FIG. 2 is a perspective view of an exemplary exclusion device, according to an embodiment of the invention.

FIG. 3 is a perspective view of an exemplary exclusion device, according to an embodiment of the invention.

FIG. 4 is an elevated perspective view of the distal end of the exemplary exclusion device of FIG. 3.

FIG. 5 is a perspective view of an exemplary exclusion device, according to an embodiment of the invention.

### DETAILED DESCRIPTION

Coronary artery bypass surgery is the largest addressable market segment for concomitant atrial fibrillation (Afib) treatment in cardiac surgery patients. This large market segment presents a significant opportunity to improve patient care with both surgical ablation and left atrial appendage (LAA) exclusion. A graft friendly exclusion device addresses both patient and customer needs.

Additionally, the largest addressable market for surgical ablation and left atrial appendage exclusion is cardiac surgery patients with no history of atrial fibrillation, but who are at a high risk of atrial fibrillation and stroke. These patients may be treated with the EnCompass™ Clamp from AtriCure® and LAA exclusion with the AtriClip™ device. Many of these patients will be coronary artery bypass graft (CABG) patients. A CABG-friendly LAA exclusion device is a long-awaited complement to CABG procedures.

LAA exclusion devices are designed to isolate and seal off the left atrial appendage (LAA). This procedure is often performed concomitantly on patients undergoing other cardiac surgeries, such as valve repair, coronary artery bypass grafting (CABG), or procedures to treat atrial fibrillation such as the Maze procedure.

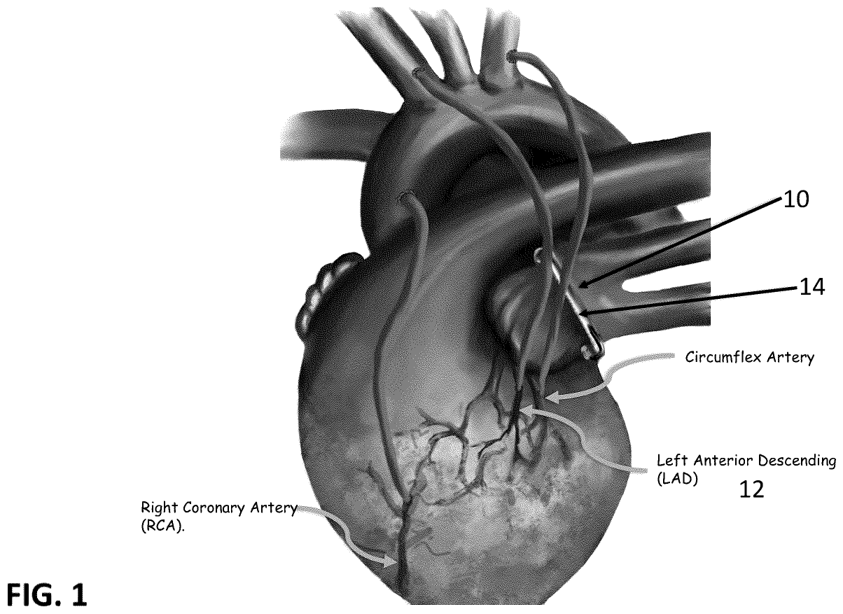

Cardiac surgery involves diverse patient anatomy and surgical techniques. Some grafts, such as the obtuse marginal (OM) branch of the circumflex coronary artery, which supplies blood to the left ventricle's side wall, may terminate near or pass in close proximity to the left atrial appendage (LAA) 10, as shown in FIG. 1. Another common graft is the Left Internal Mammary Artery (LIMA) to the Left Anterior Descending (LAD) artery 12. It has been speculated that the presence or proximity of an LAA exclusion device 14 might accelerate tissue growth, potentially leading to bypass grafts adhering to the exclusion device prematurely. This early adhesion may place undue stress on the new graft before it has fully stabilized.

Some surgeons may opt not to use an LAA exclusion device during certain CABG procedures due to concerns that the exclusion device may interfere with the bypass graft. Others may modify their surgical approach to accommodate the exclusion device by creating longer grafts and routing them around and further away from the exclusion device. However, these adjustments may counteract the primary goals of CABG, which aim for shorter, straighter bypass grafts to optimize laminar blood flow. Achieving these goals is believed to enhance the bypass grafts' longevity and patency.

Accordingly, desirable attributes of a CABG-friendly LAA exclusion device, i.e., clip, may include
(1) a very low profile, enabling the bypass graft to naturally settle into place without interference from the exclusion clip;
(2) minimal and insignificant contact with a CAB conduit, preventing erosion, stenosis, kinking, tethering, or other disruptions to the graft;
(3) minimal disruption to the bypass graft when the bypass graft is in close proximity to the exclusion device; and
(4) compatibility with saphenous vein grafts (SVGs) and arterial conduits, allowing them to be positioned on the exclusion device without concerns of conduit disruption.

The inventive exclusion device described herein includes features which promote integration with cardiac tissue at the contact surface of the exclusion device and also incorporates strategically located areas on the exclusion device which inhibit or discourage the unintended growth of the bypass grafts into or onto the exclusion device.

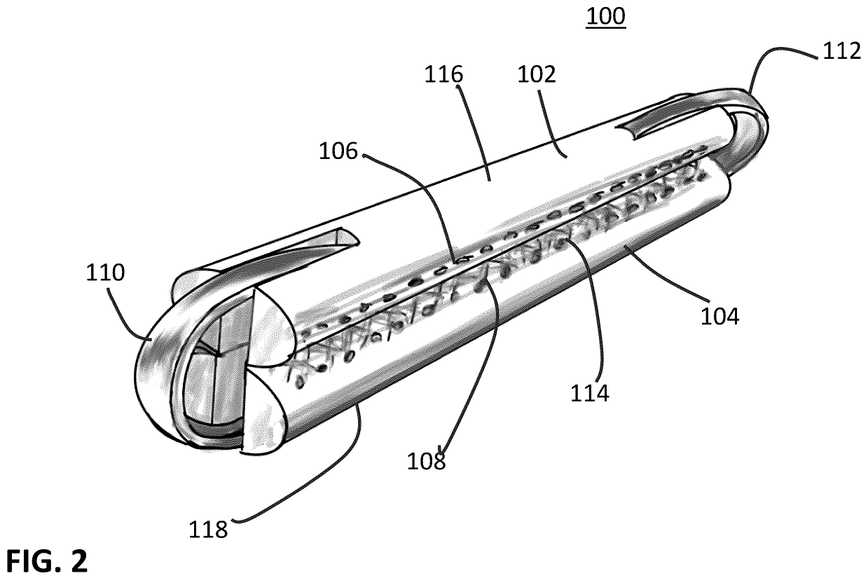

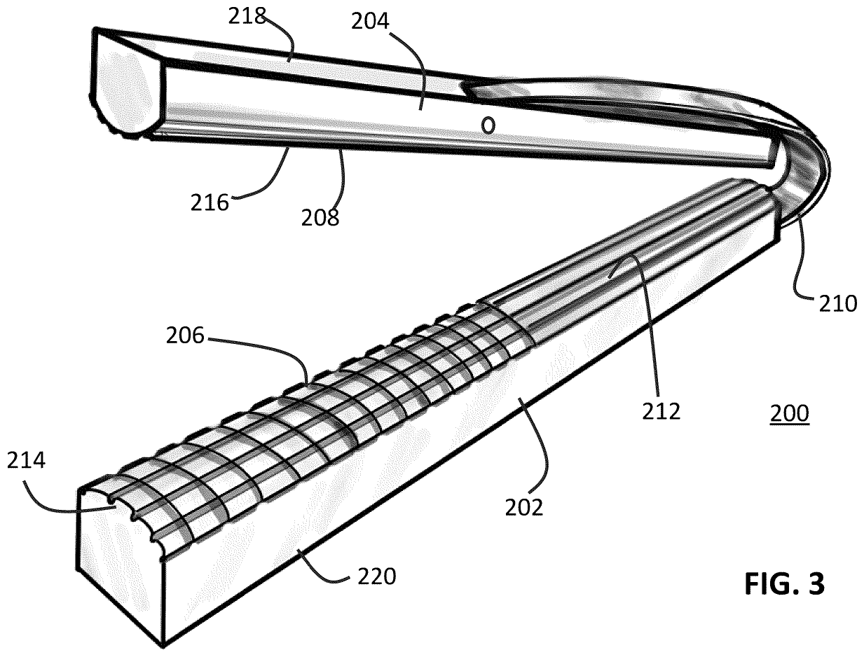

The exemplary exclusion device 100, e.g., AtriClip, depicted in FIG. 2 as a closed-loop design with at least one spring 110, 112 at the proximal and distal ends of the beams 102, 104, presents a concept that is applicable to all other exclusion clip styles, including V-shaped designs (see FIGS. 3-5). While many exclusion devices may be covered with polyester fabric to encourage tissue ingrowth and provide a scaffold for secure anchoring in biological tissue, e.g., LAA, complete fabric coverage may not always be an ideal arrangement. The area around the clip's tissue engagement surfaces 106, 108 that contact and bear against the targeted biological tissue, e.g., LAA, may be relatively porous 114, featuring small holes, texture, porous metal, or textile to allow or promote tissue infiltration and secure placement of the exclusion device 100. Conversely, the opposite sides or outer surfaces 116, 118 of the exclusion device's beams 102, 104 should inhibit tissue infiltration and attachment. This inhibition goal may be achieved through a polished, smooth finish or a drug-eluting treatment or coating that discourages tissue interaction. Alternatively, a polymer coating may achieve the same effect to discourage tissue ingrowth between the graft and the clip.

In a proposed embodiment, the exclusion device 100 is effectively integrated into the targeted tissue, e.g., LAA, mirroring the behavior of existing clip designs. This integration ensures secure and stable closure. However, unlike traditional designs, the proposed embodiment specifically addresses the concern of premature bypass graft attachment. By precisely controlling the rate and extent of tissue ingrowth, it minimizes the risk of the bypass graft adhering to the clip prematurely or contrary to bypass blood flow. This crucial aspect minimizes potential complications such as bypass graft occlusion or damage, ultimately optimizing graft function and improving patient outcomes.

The exclusion devices and tools described herein may be constructed generally as described in U.S. Pat. No. 12,232,743; U.S. provisional patent application Ser. No. 63/868,020, filed Aug. 21, 2025; U.S. provisional patent application Ser. No. 63/797,539, filed Apr. 30, 2025; U.S. provisional patent application Ser. No. 63/968,008, filed Jan. 26, 2026; as well as U.S. Pat. No. 11,389,175, the disclosures of which are incorporated herein by reference.

According to the inventive exclusion device 100 disclosed and described herein, an exclusion device 100 designed to exclude the left atrial appendage (LAA) incorporates one or more dual-function beams 102, 104. The tissue engagement areas 106, 108 of the beams 102, 104 near or against the LAA contact surface are designed to encourage tissue ingrowth, facilitating secure clip anchoring. The tissue engagement areas 106, 108 of the beams 102, 104 face each other and are biased toward each other by one or more springs 110, 112. The tissue engagement areas 106, 108 may feature surface texture, holes, partial fabric/textile coverage, or porous metal 114 to enhance tissue integration. One or more of the outward-facing portions 116, 118 of the exclusion device 100 may be in close proximity to a bypass graft, as presented in FIG. 1. Accordingly, the outward-facing portions 116, 118 are engineered to discourage tissue ingrowth. This may be achieved by polishing the surfaces 116, 118 to a finish smoother than 32 microinches. The finish may be further refined through electropolishing to minimize or eliminate microscopic pores. In the alternative, the outward-facing portions 116, 118 of the beams may incorporate a drug-eluting coating or a polymer coating/attached component to further inhibit tissue interaction and prevent unwanted adhesion. In another variation of the exclusion device, only one of the outward-facing portions 116, 118 is engineered to discourage tissue ingrowth. In this instance, the outward-facing portion 116, 118 that is engineered to discourage tissue ingrowth is oriented away from the patient's heart. The outward-facing portion 116, 118 that is against the patient's heart is unlikely to come into contact or close proximity with a coronary artery bypass graft (CABG). In a further advantageous arrangement, the exclusion device may include a very low-profile configuration. This enables the CABG to naturally settle into place without interference from the exclusion device 100.

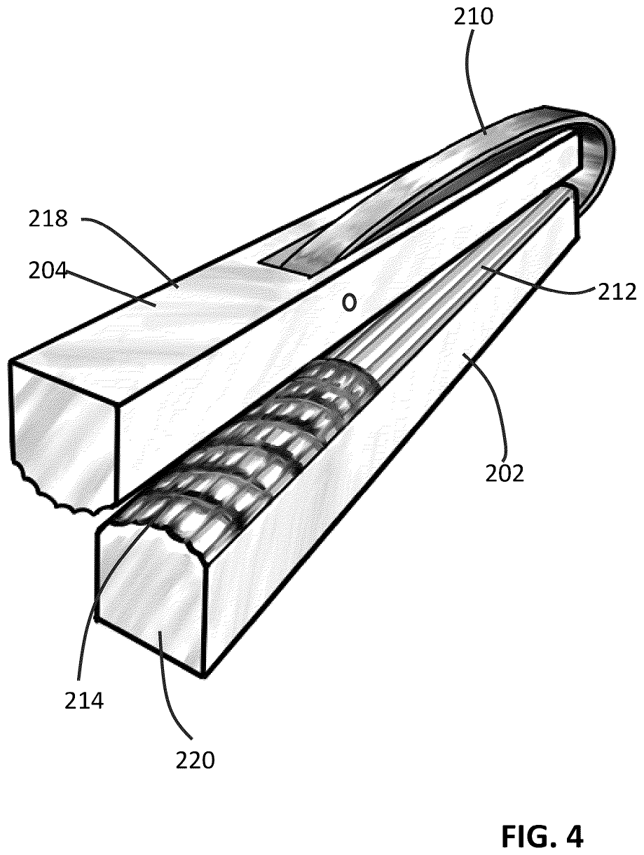

FIGS. 3-4 present perspective views of an exemplary exclusion device 200, according to an embodiment of the invention. According to the inventive exclusion device disclosed and described herein, an exclusion device 200 designed to exclude the left atrial appendage (LAA) incorporates one or more dual-function beams 202, 204. The tissue engagement areas 206, 208 of the beams 202, 204 near or against the LAA contact surface are designed to encourage tissue ingrowth, facilitating secure exclusion device anchoring. The tissue engagement areas 206, 208 of the beams 202, 204 face each other and may be biased toward each other by a single spring 210. The tissue engagement areas 206, 208 may feature surface texture, holes, partial fabric/textile coverage, or porous metal 212, 214, 216 to enhance tissue integration. One or more of the outward-facing portions 218, 220 of the exclusion device 200 may be in close proximity to a bypass graft, as presented in FIG. 1. Accordingly, the outward-facing portions 218, 220 may be engineered to discourage tissue ingrowth. This may be achieved by polishing the outward-facing surfaces 218, 220 to a finish smoother than 32 microinches, which may be further refined through electropolishing to minimize or eliminate microscopic pores. In the alternative, the outward-facing portions 218, 220 of the beams 202, 204 may incorporate a drug-eluting coating or a polymer coating/attached component to further inhibit tissue interaction and prevent unwanted adhesion. In another variation of the exclusion device 200, only one of the outward-facing portions 218, 220 is engineered to discourage tissue ingrowth. In this instance, the outward-facing portion 218, 220 that is engineered to discourage tissue ingrowth is oriented away from the patient's heart. The outward-facing portion 218, 220 that is against the patient's heart is unlikely to come into contact or close proximity with a coronary artery bypass graft (CABG). In a further advantageous arrangement, the exclusion device 200 may include a very low-profile configuration. This enables the CABG to naturally settle into place without interference from the exclusion device 200.

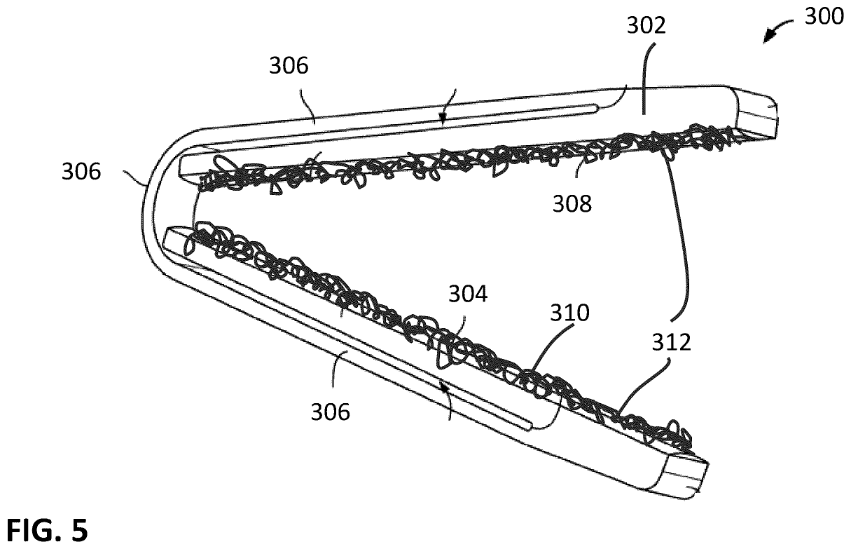

FIG. 5 presents a third type of exclusion device 300 having beams 302, 304 which are biased closed by a spring 306. A single spring is shown, but there may be one or more springs at each end of the beams 302, 304. The exclusion device 300 is presented with an alternative surface for tissue engagement areas 308, 310 of the beams 302, 304 that face each other and are biased together by spring 306. The third embodiment of the exclusion device 300 is presented to demonstrate that the alternative surface of the tissue engagement areas 308, 310 may be a nonwoven fabric 312, such as an electrospun fabric. The electrospun fabric may comprise one or more of polyethylene terephthalate (PET) yarn, thermoplastic polyurethane (TPU), polycaprolactone (PCL), and poly(lactic-co-glycolic acid) (PLGA), but any biocompatible fiber that is able to be deposited by electrospinning may be used.

The nonwoven fabric 312 may be applied the exclusion devices 100, 200 described above and to tissue engagement areas 106, 108, 206, 208 of the beams 102, 104, 202, 204 that face each other and are biased toward each other by the one or more springs 110, 112, 210.

The areas of the exclusion device 300 where the electrospinning depositions may be made includes the tissue engagement faces 308, 310 of the beams 302, 304, and any area facing the patient's heart and/or facing away from any coronary artery bypass graft. Electrospinning permits the nonwoven fabric 312 to be deposited and fixed to the exclusion device 300 with a high degree of precision and selectivity.

A number of embodiments have been described. Nevertheless, it will be understood by one or ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations can be provided, or steps or operations can be eliminated or omitted from the described methods or processes to achieve the desired results. Moreover, any components or parts of any apparatus or systems described in this disclosure or depicted in the figures can be removed, eliminated, or omitted to achieve the desired results. In addition, certain components or parts of the systems, devices, or apparatus shown or described herein have been omitted for the sake of succinctness and clarity.

Accordingly, other embodiments are within the scope of the disclosure and the specification and/or drawings can be regarded in an illustrative rather than a restrictive sense.

Each of the individual variations or embodiments described and illustrated herein has discrete components and features which can be readily separated from or combined with the features or any of the other variations or embodiments. Modifications can be made to adapt a particular situation, material, composition or matter, process, process act(s) or step(s) to the objective(s), spirit, or scope of the present invention.

Methods recited herein can be carried out in any order of the recited events that is logically possible, as well as the recited order of events. Moreover, additional steps or operations can be provided or steps or operations can be eliminated to achieve the desired result.

Furthermore, where a range of values is provided, every intervening value between the upper and lower limit of that range and any other stated or intervening value in that stated range is encompassed within the invention. Also, any optional feature of the inventive variations described can be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter can conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a, an,” “said” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use or a “negative” limitation. Unless defined otherwise, all technical and scientific terms used herein have the same meaning as commonly understood by one or ordinary skill in the art to which this invention belongs.

Reference to the phrase “at least one of, when such phrase modifies a plurality or items or components (or an enumerated list or items or components) means any combination or one or more or those items or components. For example, the phrase “at least one or A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part, section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms or degree such as “substantially”, “about” and “approximately” as used herein mean the specified value or the specified value and a reasonable amount of deviation from the specified value (e.g., a deviation of up to 1%, %, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents or the variations or embodiments described herein. Further, the scope of the disclosure fully encompasses other variations or embodiments that can become obvious to those skilled in the art in view of this disclosure.

1. An exclusion device comprising:
a pair of elongated beams, each of the beams including a tissue engagement surface and an outer surface, the tissue engagement surfaces facing each other, wherein the pair of elongated beams each have a dominant dimension in a longitudinal direction;
at least one spring coupled to the pair of elongated beams and biasing the beams toward each other, wherein the tissue engagement surfaces include at least one of a porous surface, a textured surface, or a textile.

2. The exclusion device of claim 1, wherein the porous surface comprises a plurality of holes.

3. The exclusion device of claim 1, wherein the textured surface comprises a plurality of a linear pattern, a crosshatched pattern, ridges, checkering, or bumps.

4. The exclusion device of claim 1, wherein the textile comprises at least one of a nonwoven fabric selected from polyethylene terephthalate (PET) yarn, thermoplastic polyurethane (TPU), polycaprolactone (PCL), and poly(lactic-co-glycolic acid) (PLGA).

5. The exclusion device of claim 1, wherein the tissue engagement surfaces include a convex profile extending in the longitudinal direction and including a first longitudinal apex.

6. The exclusion device of claim 1, wherein the at least one spring is configured to apply a uniform closing force along the first and second tissue engaging surfaces.

7. The exclusion device of claim 1, wherein the texturized tissue engagement surfaces are interlocking.
